(19) United States
(12) Patent Application Publication
(10) Pub. No.: US 2021/0023154 A1
(43) Pub. Date: Jan. 28, 2021

(54) BIOLOGICAL MICROBIAL DRUG FOR TREATING SKIN DISEASES, AND PREPARATION METHOD AND APPLICATION THEREOF

(71) Applicant: Siguo SUN, Jining (CN)

(72) Inventor: Siguo SUN, Jining (CN)

(21) Appl. No.: 16/562,453

(22) Filed: Sep. 6, 2019

(30) Foreign Application Priority Data
Jul. 25, 2019 (CN) 201910677050.6

(51) Int. Cl. A61K 36/07; A61K 36/889; A61K 36/481; A61K 36/79; A61K 36/296; A61K 36/46; A61K 36/75; A61K 36/815; A61P 17/06

(52) U.S. Cl. CPC A61K 36/07; A61K 36/889; A61K 36/481; A61K 36/79; A01G 24/25; A61K 36/46; A61K 36/75; A61K 36/815; A61P 17/06; A61K 36/296

(57) ABSTRACT

A biological microbial drug for treating skin diseases as well as a preparation method and application thereof. The biological microbial drug includes Pleurotus ostreatus cultivated in a medium added with a Traditional Chinese Medicine (TCM) combination, and the TCM combination includes Sangusis draconis, Astragalus membranaceus, Schisandra chinensis, Herba epimedii, Eucommia ulmoides, Chinese wolfberry and Cortex dictamni. The dry biological microbial drug can be brewed like tea or boiled with water, and is non-bitter, non-toxic, tasteless, free of side effects, green, natural, and suitable for the treatment of psoriasis, eczema, urticaria and neurodermatitis.

### CROSS REFERENCE TO THE RELATED APPLICATIONS

This application is based upon and claims priority to Chinese Patent Application No. 201910677050.6, filed on Jul. 25, 2019, the entire contents of which are incorporated herein by reference.

### TECHNICAL FIELD

The invention belongs to the field of Chinese herbal medicines, and particularly relates to a biological microbial drug for treating skin diseases, and a preparation method and application thereof.

### BACKGROUND

As the first physiological defense line and the organ with the largest area of the human body, the skin is constantly involved in the functional activities of the human body, maintaining the unity and harmony between the body and natural environment. Therefore, abnormal condones of the body can also be reflected on the skin surface. The impairment to the physiological function of the skin may cause dermatosis (skin disease). The skin disease is a general term for diseases of the skin and skin accessory organs. It is a common disease by a wide variety and complicated causes. Common skin diseases include eczema, dermatitis, impetigo, urticaria, pruritus, and psoriasis, etc.

The psoriasis, also known as psoriasis vulgaris, white tinea, and pine bark tinea, is a common chronic inflammatory skin disease with characteristic skin lesions and easy recurrence. There are a large number of patients, with the number ever increasing every year. The cause of this disease has not yet been clarified. According to modern medicine, the occurrence of psoriasis may be related to genetics, immune system, endocrine system, psychological, infection, lifestyle, drug, and environmental factors. From the beginning, it is manifested by inflammatory red papules, about the size of millet to mung bean, then merges or gradually enlarges into a brown-red plaque with clear boundaries, surrounded by inflammatory redness, obvious basal infiltration, and covered with multiple layers of dry grayish white or silvery white scales, and it may form to a large map or cover the whole body in severe cases. If the surface scales are gently scraped off, a layer of translucent film is gradually exposed, which is light red and shiny, and this is called the film phenomenon. If the film is removed further, there will be small bleeding points, called punctiform bleeding. The white scales, shiny films, and spotted hemorrhage are the triad for the diagnosis of psoriasis. It is prone to occurring on the scalp, limbs and the back, and most patients have serious symptoms in winter and less symptoms in summer. The psoriasis can be divided into three periods from the occurrence to final retreat: \textcircled{1} progression: new rashes appear constantly, and old ones continue to expand, scaly thick, with obvious inflammation, obvious itching, and increased skin sensitivity; \textcircled{2} quiescent period: no new rashes appear, and old rashes are not easy to subside; \textcircled{3} regression: the inflammation subsides, and the scales decrease, and the rashes shrink and flatten, and light halos appear around them, which may leave temporary hypopigmentation or sedimentation.

Although the psoriasis does not directly threaten life, it is intractable and difficult to treat. It has a direct impact on the physical and mental health of the patients, and it is still listed as an important research topic and one of diseases for key prevention and treatment in dermatology department across the world, so it is also called “immortal cancer.”

In existing technology, there are many drugs for treating the psoriasis. However, the existing Chinese medicine treatment usually has a long treatment course, without an obvious effect, and the long-term decoction is troublesome, and the bitterness of the traditional Chinese medicine makes it very uncomfortable to take. Western medicine includes the drugs taken orally, such as methotrexate, vitamin A acids, hormones, immunosuppressive agents, etc., and the drugs for topical use, such as corticosteroid hormone ointment. By means of injecting hormones, transfer factors or anticancer drugs, the clinical cure can be quickly achieved, but the patients may experience continuous outbreaks within 10-30 days after the discharge from the hospital, which may be even more severe than the psoriasis before hospitalization. Moreover, the dose required for retreatment after the drug withdrawal will higher and higher, which may cause drug toxic and side effects and hormone dependence, so only the symptoms are not cured, instead of the disease. The recurrence of the disease made many patients lose confidence, and be painful inside and even suicidal. This is the main reason why the whole world refers to psoriasis as “immortal cancer.”

### SUMMARY

The above situation has stimulated our confidence and determination to completely cure the psoriasis. In order to find a faster, better and more effective cure method, after numerous trials, repeated demonstrations, free trials, etc., we finally succeeded in developing a biological microbial drug—Pleurotus ostreatus, which is brewed like tea or boiled with water and has the characteristic of being non-bitter, non-toxic, free of obvious Chinese medicinal taste and side effects, green and natural, and having a significant effect in treatment of psoriasis, eczema, urticaria and neurodermatitis.

In order to achieve above objective, following technical solutions are adopted by the invention:

This invention provides a biological microbial drug for treating skin diseases. The biological microbial drug includes Pleurotus ostreatus cultivated in a Pleurotus ostreatus medium added with a TCM combination, and the TCM combination includes Sangusis draconis, Astragalus membranaceus, Schisandra chinensis, Herba epimedii, Eucommia ulmoides, Chinese wolfberry and Cortex dictamni.

Preferably, the weight ratio of the TCM combination to the Pleurotus ostreatus medium is 89.5-110.5: 162-324 (more preferably 100:162), and the Pleurotus ostreatus medium added with the TCM combination has a water content of 55-65% and a pH value of 6.5-8.

Preferably, the TCM combination includes, by weight, 3.5-6.5% of Sangusis draconis, 18.5-21.5% of Astragalus membranaceus, 13.5-16.5% of Schisandra chinensis, 8.5-11.5% of Herba epimedii, 3.5-6.5% of Eucommia ulmoides, 28.5-31.5% of Chinese wolfberry and 13.5-16.5% of Cortex dictamni.

More preferably, the TCM combination includes, by weight, 5% of Sangusis draconis, 20% of Astragalus membranaceus, 15% of Schisandra chinensis, 10% of Herba epimedii, 5% of Eucommia ulmoides, 30% of Chinese wolfberry and 15% of Cortex dictamni.

Preferably, the Pleurotus eryngii medium includes cottonseed husk/broadleaf tree sawdust/corn core/bean straw powder/peanut shell/bamboo, soybean cake powder, maize meal, bran, lime powder, gypsum powder and brown sugar/white sugar, with a correspondingly weight ratio of 100-200:10-20:20-40:20-40:6-12:4-8:2-4.

More preferably, in the Pleurotus ostreatus medium, the weight ratio of cottonseed husk/broadleaf tree sawdust/corn core/bean straw powder/peanut shell/bamboo, soybean cake powder, maize meal, bran, lime powder, gypsum powder and brown sugar/white sugar is 100:10:20:20:6:4:2.

The invention further provides a method for preparing the biological microbial drug for treating skin diseases, including the following steps:

1) A Pleurotus ostreatus culture medium added with a TCM combination and having a water content of 55-65% and a pH value of 6.5-8 is prepared and sterilized, wherein the TCM combination includes Sangusis draconis, Astragalus membranaceus, Schisandra chinensis, Herba epimedii, Eucommia ulmoides, Chinese wolfberry and Cortex dictamni, and the Pleurotus ostreatus medium includes cottonseed husk/broadleaf tree sawdust/corn core/bean straw powder/peanut shell/bamboo, soybean cake powder, maize meal, bran, lime powder, gypsum powder and brown sugar/white sugar, and the weight ratio of the TCM combination to the Pleurotus ostreatus medium is 89.5-110.5:162-324;

2) Under sterile conditions, the strain of Pleurotus ostreatus is inoculated by 4-9%, wherein the incubation temperature is below 30 degrees, and the humidity is 60-70%;

3) After the mushrooms appear, the humidity is kept at 80-95%;

When growing the harvesting standard, the Pleurotus ostreatus s harvested and dried in time to obtain the biological microbial drug.

4) Preferably, the Pleurotus ostreatus culture medium added with the TCM combination includes, by weight, 3.5-6.5 parts of Sangusis draconis, 18.5-21.5 parts of Astragalus membranaceus, 13.5-16.5 parts of Schisandra chinensis, 8.5-11.5 parts of Herba epimedii, 3.5-6.5 parts of Eucommia ulmoides, 28.5-31.5 parts of Chinese wolfberry and 13.5-16.5 parts of Cortex dictamni, and 100-200 parts of cottonseed husk/broadleaf tree sawdust/corn core/bean straw powder/peanut shell/bamboo crumbs, 10-20 parts of soybean cake powder, 20-40 parts of maize meal, 20-40 parts of bran, 6-12 parts of lime powder, 4-8 parts of gypsum powder and 2-4 parts of brown sugar/white sugar.

More preferably, the Pleurotus ostreatus culture medium added with the TCM combination includes, by weight, 5 parts of Sangusis draconis, 20 parts of Astragalus membranaceus, 15 parts of Schisandra chinensis, 10 parts of Herba epimedii, 5 parts of Eucommia ulmoides, 30 parts of Chinese wolfberry and 15 parts of Cortex dictamni, and 100 parts of cottonseed husk/broadleaf tree sawdust, 10 parts of soybean cake powder, 20 parts of maize meal, 20 parts of bran, 6 parts of lime powder, 4 parts of gypsum powder and 2 parts of brown sugar/white sugar.

The invention further provides an application of the biological microbial drug for treating skin diseases in the preparation of the medicament for treating such skin diseases.

Preferably, the skin diseases include psoriasis, eczema, urticaria, and neurodermatitis.

The invention also provides a method for treating skin diseases with the biological microbial drug.

Preferably, the method includes brewing the dried Pleurotus ostreatus as tea or boil the dried Pleurotus ostreatus in water, and take the dried Pleurotus ostreatus once a day.

The beneficial effects of the invention are as follows:

1. Biological microbial drug—as the name suggests, the Pleurotus ostreatus is the product of affinal drug and diet used to radically cure psoriasis, eczema, urticaria and neurodermatitis, which is produced by inoculating the strain of Pleurotus ostreatus to the medium containing a variety of Chinese medicinal materials for culture, and the Pleurotus ostreatus can extract the nutrients, and the features and efficacy of each Chinese medicinal material are transferred to the Pleurotus ostreatus by adjusting the humidity, pH value and scattered light for meticulous culture for 180 days during the growth process. The product is used to treat the psoriasis mainly based on the mechanism of warming the kidney to tonify yang, regulating qi to strengthen the spleen and dehumidification, astringing sores and resolving carbuncle, and detoxification and promoting tissue regeneration. Among the 7 Chinese medicinal materials used in the culture medium, the Sangusis draconis is flat, sweet and salty, with the effects of promoting blood circulation and relieving pain, relieving blood stasis and hemorrhage, and astringing sores and resolving carbuncle; the Astragalus membranaceus is sweet and slightly warm in nature, with the effects of tonifying qi and strengthening exterior, arresting sweating and relieving depletion, astringing sores and resolving carbuncle and inducing diuresis to alleviate edema; the Schisandra chinensis is sour, sweet, and warm in nature, with the effects of astringency and arresting discharge, supplementing qi and promoting the production of body fluid, tonifying the kidney and calming the heart, astringing the lung to stop cough, which can be used to treat deficiency syndrome of the lung and chronic cough, spontaneous sweating and night sweating, thirst, and insomnia, etc.; the Herba epimedii is spicy and warm in nature, with the effects of tonifying kidney yang, strengthening the bones and muscles, and expelling wind-damp; The Eucommia ulmoides is sweet and warm in nature, with the effects of tonifying the liver and kidney, strengthening tendons and bones, coordinating Chong and conception vessels, and stabilizing meridians and miscarriage prevention, and it can be used to treat pain in waist and lower extremities or aching and limp caused by kidney yang deficiency; the Chinese wolfberry is sweet and flat, with the effect of nourishing yin and tonifying the kidneys and benefiting the essence, and it also has the functions of enhancing immunity, anti-aging and anti-tumor; the Cortex dictamni is bitter and cold in nature, with the effects of clearing heat and dampness, dispelling the wind and detoxifying, which can be used treat various dermatitis and eczema caused by fungal infection.

2. Biological microbial drug—the treatment of psoriasis with Pleurotus erythema can better promote the skin lesions to retreat and achieve ideal treatment efficacy, which is attributable to the fact that the components of biological microbial drug penetrate into human body, improve internal environment of the organism, control the synthesis and proliferation of the cells controlling the disease, and thus greatly improve the patient's own immunity, dispel all the viruses from the body, and promote the metabolism of epidermal cells to turn back normal.

3. Efforts should be made to regulate the 5 internal organs of the human body in the treatment of psoriasis. TCM pays more attention to the treatment of “the root causes” rather than the“ the symptoms” in Western medicine. “Identifying the essence”, so-called “treating the root causes”, and dialectical treatment can effective eliminate the lesions and cure the disease by regulating 5 organs of human body. Specifically, it brings out full play of the efficacy, promote the balance of internal environment of human body, restore normal metabolism of the skin from the inside to the outside, and eventually cure the psoriasis.

4. Different from other TCM formulations, the biological microbial drug—Pleurotus ostreatus of this invention absorbs the essence of various TCM materials and different nutrients of Pleurotus ostreatus, without TCM taste, but only slight taste of mushroom, so it is green and natural without toxic and side effects. It is brewed as tea or boiled with water, and taken once either before or after meal every day. The cost is low, and as long as the treatment is adhered to, 97% of the patients with psoriasis can be cured, so it is green therapy worth trying.

5. Except psoriasis, this product has significant effects in treatment of eczema, urticaria and neurodermatitis.

### BRIEF DESCRIPTION OF THE DRAWINGS

FIG. 1 is the comparative photos of No. 1 patient before and after treatment with the biological microbial drug of the invention, (a) before treatment, and (b) after treatment.

FIG. 2 is the comparative photos of No. 2 patient before and after treatment with the biological microbial drug of the invention, (a) before treatment, and (b) after treatment.

FIG. 3 is the comparative photos of No. 3 patient before and after treatment with the biological microbial drug of the invention, (a) before treatment, and (b) after treatment.

FIG. 4 is the comparative photos of No. 4 patient before and after treatment with the biological microbial drug of the invention, (a) before treatment, and (b) after treatment.

### DETAILED DESCRIPTION OF THE EMBODIMENTS

The technical solutions of the invention are clearly and completely described below with reference to embodiments. Clearly, the embodiments in the following description are only illustrative ones of the invention. Based on these illustrative embodiments, all other embodiments obtained by those ordinarily skilled in the field without creative efforts are all within the protection scope of the invention.

### Embodiments

(I) Inoculation and Culture of Pleurotus ostreatus Strain, Original Strain and Production Strain

1. (Grade I) Inoculation and Culture of Parent Strain

1) Strain: Pleurotus ostreatus c Gm CC 5.729, purchased from the Institute of Microbiology, Chinese Academy of Sciences. Other strains of Pleurotus ostreatus may also be used in the invention, and the invention has no limitation in this aspect.

2) Medium Formula:

200 g of wheat grains, 20 g of glucose, 3 g of potassium dihydrogen phosphate, 1.5 g of magnesium sulfate, 10 g of peptone, 4 tablets of vitamin B, 20 g of agar, and 1000mL of water.

3) Inoculation

a) First the wheat grainswith soft fire for 20 minutes, and then filter.

b) Add water and heat, and then boil the agar, glucose, magnesium sulfate, monopotassium phosphate, peptone, and vitamin B until complete dissolution. The mixed solution is measured with measuring glass to check if it is 1000 mL; if it is not sufficient, make up with boiling water.

c) Dispense the medium into tubes with 10-12 mL of liquid in each tube. Pay attention not to allow the medium to get on the test tube when dispensing, which may otherwise contaminate the cotton plug with contaminated bacteria. Then put on the cotton plug, tighten moderately, tie up the test tubes by 10/bundle, wrap the cotton plug and test tube opening with kraft paper, put it into the pressure cooker and sterilize for 40 minutes at the pressure of 1.5 kg, turn off the fire, and wait until the pressure gauge is zero before exhaustion and open the cover, and take out the test tubes and place on the table inclined, wherein the length of the slope is ⅓ that of the tubes.

d) Start inoculation when the temperature of the test tubes drop below 30 degrees, and carry out inoculation under strict aseptic conditions (e.g., ultra-clean work bench and aseptic chamber).

4) Culture

After being inoculated into the test tube, the strain placed in a 25° C. incubator and is cultured for 7-12 days. After test tube is full of the mycelium and checked without any other bacteria, the second-class original strain is inoculated.

2. (Grade II) Inoculation and Cultivation of Original Strain p 1) Formula of Culture Materials:

20 kg of broadleaf tree sawdust, 20 kg of corn cob, 5 kg of soybean cake powder, 5 kg of maize meal, 500 g of sugar, 250 g of magnesium sulfate, 250 g of monopotassium phosphate, 500 g of gypsum powder, and 500 g of lime powder.

2) Inoculation and Culture

Add 65% of water to the culture materials (i.e., the water content of the medium is 65%), mix the water and the culture materials, dispense the culture materials into strain bottles or the strain bags, and sterilize the culture materials thoroughly with conventional method. When the temperature drops to 30° C., transfer the culture materials to the inoculation room or inoculation chamber (all sterile) and inoculate the parent strain. After inoculation, culture at 25-30° C. for 25-30 days, and the strain can be used after being tested as bacteria free.

3. (Grade III) Inoculation and Cultivation of Production Strain

In the same way as the original strain produced, the times of production transference can only be increased to obtain sufficient amount of strain for production.

(II) Biological Microbial Drug—Inoculation of Pleurotus ostreatus

1. Preparation of the Medium:

5 parts of Sangusis draconis (powder), 20 parts of Astragalus membranaceus (granules), 15 parts of Schisandra chinensis (twig powder/trunk sawdust), 10 parts of Herba epimedii (powder), 5 parts of Eucommia ulmoides (granules/powder), 30 parts of Chinese wolfberry (trunk sawdust) and 15 parts of Cortex dictamni trunk (powder); there are 100 parts of TCM materials in total.

In addition, 100 parts of cottonseed husk/broadleaf tree sawdust, 10 parts of soybean cake powder, 20 parts of maize meal, 20 parts of bran, 6 parts of lime powder, 4 parts of gypsum powder and 2 parts of brown sugar.

The water content is 65% and the pH is 6.5-8.

The formula is the optimal one of the invention. The content of each component in the TCM combination, the composition and content in the Pleurotus ostreatus medium, and the added amount of the TCM combination in the Pleurotus ostreatus medium can be adjusted by those skilled in this field within a proper range according to practical requirements, but the invention is not limited thereto.

The optimal formula of the TCM combination is finally determined by the applicant of the invention after numerous times of experiment. If the active ingredients released by various TCM materials are too high, the emergency of mycelium of Pleurotus ostreatus will be inhibited, and even the culture materials will undergo acid deterioration, and contamination by other bacteria, and the active ingredients become low, and the grown Pleurotus ostreatus has no TCM effect or a slight TCM effect.

It is noteworthy that in the TCM materials selected in this application, Schisandra chinensis and Chinese wolfberry used are in the form of twig powder and trunk sawdust respectively, instead fruits. This is because the applicant found during the test that the organic acid content in their fruits is too high, causing complete failure of the culture, while after smashing of Chinese wolfberry and Schisandra chinensis trees into scraps (sawdust), the nutrition provided by these “offals” can not only achieve a corresponding curative effect of the cultured Pleurotus ostreatus, without bringing negative impact to the culture.

2. Sterilization for Bag Package

1) Stack all raw materials together on the concrete floor, rack over for 3 times for drying, stir while adding tap water, so that the raw materials can fully absorb the water. After the raw materials are mixed, grip tightly with the hand until 3-5 drops come out of the culture medium, measure if the pH value is qualified: if the pH value is low, add a small amount of lime powder for adjustment;

2) After the completion of step 1), stack the culture materials in a trapezoidal shape, punch numerous holes from the top, so that the culture materials are ventilated up and down, and then cover with a plastic film, and begin to fill in bags 24 hours later;

3) Use 20*42 cm plastic bags, wherein each bag can hold 1.8 kg of dry substance. After filling the bag, sterilize immediately in autoclave (100 kPa, 121° C.) for 3 hours or at atmospheric pressure (100° C.) for 16 hours. In both sterilization at normal pressure or high pressure, there should be designated person for observation until finally achieving the effect of completely killing all bacteria.

3. Inoculation

Biological microbial drug—the Pleurotus ostreatus bag is relatively large, so it should not be inoculated in the sterile chamber, but generally inoculated in the inoculation room or simple inoculation curtain.

1) First clean the inoculation room or curtain, and transfer the sterilized bags, strain, tools, and alcohol lamp into the inoculation room or curtain, and fumigate and sterilize by 4 g of smoke disinfectant per cubic meter, and keep the door and window sealed for 24 hours.

2) The next morning, the staff quickly enters the inoculation room or curtain to completely disinfect all the tools and hands with 75% alcohol, and then open the bag and inoculate by the inoculation amount of 5-8% (generally 500 g of bacterial strain for 20 bags). The inoculation amount should not be too large or too small: if a too large inoculation amount may easily cause early mushrooming, thus inhibiting the growth of mycelium; a too small inoculation amount may cause slow growth rate, affecting the time of mushrooming, and easily cause contamination of other bacteria.

3) About 2 days after inoculation, the mycelium begins to germinate; after 30 days, the whole bag is full of hyphae, white and thick, with good elasticity. During the period of mushrooming, the bacteria bag should be controlled below 30° C. (optically 20-28° C., relative humidity of 60-70%), and the contaminated bags should be taken out in time for destruction.

4. Mushrooming Management

Biological microbial drug—After the mushroom buds appear on the Pleurotus ostreatus bag, pay attention to the ventilation and humidification. Both ends of the bacteria bags should be opened to facilitate ventilation, and the relative humidity in the air should be increased by spraying water to the ground, walls and spaces and maintained at about 80-95%. It is forbidden to spray water directly into the mushroom buds. When the mushroom buds form the size of the soybean granules, increase the ventilation volume, and provide scattered light. As the mushrooms grow, increase the humidity in the mushroom room. Perform light spray, frequent spray, and fine spray to strengthen ventilation and air exchange, keep the air fresh and moist to facilitate normal growth and development of the sporocarp. At this time, control the light intensity at the standard of about 1000-1500 lux, and the carbon dioxide within 0.5%.

5. Harvesting Standard

Biological microbial drug—the harvesting standard for Pleurotus ostreatus is that the mushroom cap is fully spread, but with inwardly curled edge, and the color gradually becomes paler, and the white hair starts to appear in the concave part. Early harvesting affects the production, and late harvesting makes the edge of mushroom cap become thin, the flesh become loose, and the stalk become aged, thick and hard, thus leads to degraded quality, poor taste, and reduced weight, which affects the growth of the next generation of mushrooms or affects the mushrooming.

6. Processing

When the mushroom cap is fully spread and ready to mature, collect the whole Pleurotus ostreatus, and tear the whole mushroom into a single piece or strips for drying (control the temperature at 40° C. for the first 2 hours, then increase to 60° C. until dry). Do not dry under the sun, because the ultraviolet rays will destroy the efficacy and value of Pleurotus ostreatus.

After being dried, put in a plastic bag and seal the opening to prevent moisturizing again.

### APPLICATION EXAMPLES

The applicant provided the biological microbial drug of the invention to 60 patients with psoriasis, and the patients with psoriasis were randomized evenly into 2 groups which correspondingly take the biological microbial drug prepared in Embodiment 1 once a day; the efficacy includes “effective”, “ineffective”, and “recovered”. During the medication, other stimulant drugs should not be taken, and blood routine and urine routine examinations should be performed regularly every month.

Ineffective: the symptoms of psoriasis are significantly improved;

Ineffective: no obvious effect;

Recovered: the psoriasis symptoms disappear without recurrence.

The numbers of effective and recovered cases in clinical trial are counted in 3 months and 6 months. The results are shown in Tables 1-3, respectively.

TABLE 1
Statistics on the number of effective and recovered cases
after taking the biological microbial drug of the invention
for 3 months in the 2 groups of psoriasis patients

| Group | Cases | Effective | Ineffective | Recovered |
|---|---|---|---|---|
| Group 1 | 30 | 30 | 0 | 23 |
| Group 2 | 30 | 30 | 0 | 24 |

TABLE 2
Statistics on the number of effective and recovered cases
after taking the biological microbial drug of the invention
for 6 months in the 2 groups of psoriasis patients

| Group | Cases | Effective | Ineffective | Recovered |
|---|---|---|---|---|
| Group 1 | 30 | 30 | 0 | 28 |
| Group 2 | 30 | 30 | 0 | 30 |

TABLE 3
Statistics on the number of effective and recovered cases after
taking the biological microbial drug of the invention for 3
and 6 months respectively in the 2 groups of psoriasis patients

| | | | | Effective | Recovered |
|---|---|---|---|---|---|
| Time | Effective | Ineffective | Recovered | rate | rate |
| 3 months | 60 | 0 | 47 | 100% | 78% |
| 6 months | 60 | 0 | 58 | 100% | 97% |

It can be seen from Table 1-3 that the biological microbial drug for treating psoriasis provided by the invention can achieve a recovered rate higher than 78% and an effective rate of 100% in 3 months, which means that the biological microbial drug has a very good efficacy; it is observed in the trial that the skin of the recovered patients is restored to the original state within 30 days, without recurrence observed. The liver and kidney functions of the patient are normal during the drug administration, and no adverse injuries have been caused after taking the medicine.

In order to explain the authenticity of the effect of the invention, the treatment records of the patients taking the biological microbial drug of the invention are taken as the examples, and the consents have been obtained from the patients for the disclosure of the patient information and the informed consent forms have been signed.

Clinical Case 1

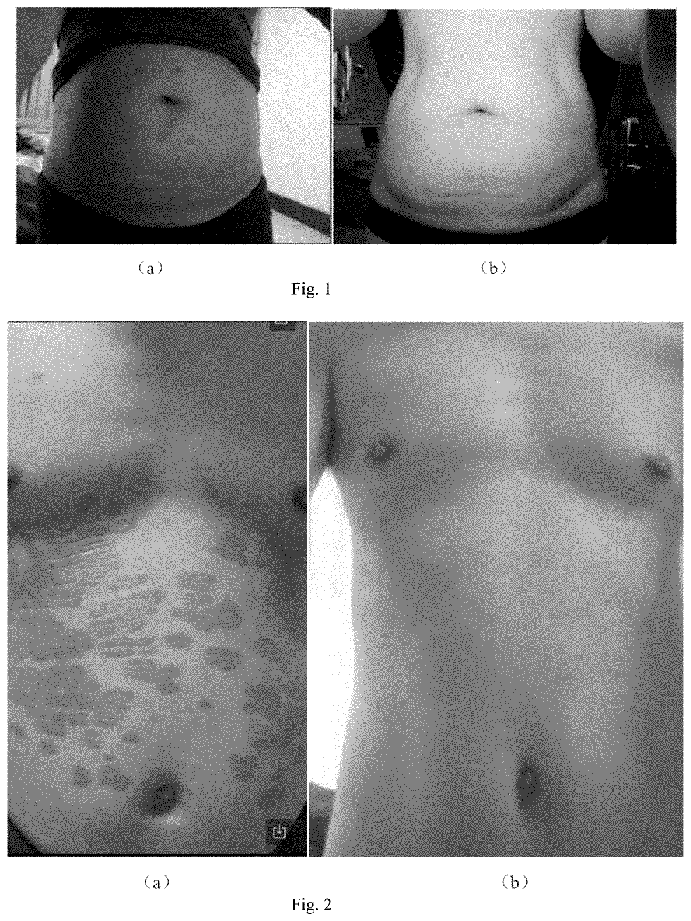

No. 1 patient, Liu * Mei, (151****7326, Building No. 3, Zhanqian Resident, Yichun District, Yichun City, Heilongjiang Province), suffered from psoriasis for 32 years, and was recovered after taking the biological microbial drug for 6 months, and completely rehabilitated after consolidation therapy for 1 month. The skin conditions before and after treatment are shown in FIG. 1.

Clinical Case 2

No. 2 patient, Cao*shun, (159****0780, Xiaoyun Village, Xiaoyun Town, Jinxiang County, Shandong Province), suffered from psoriasis for 6 years, and smooth all over the body after taking the biological microbial drug for 4 months, and was completely recovered after taking it for another 2 months. The skin conditions before and after treatment are shown in FIG. 2.

Clinical Case 3

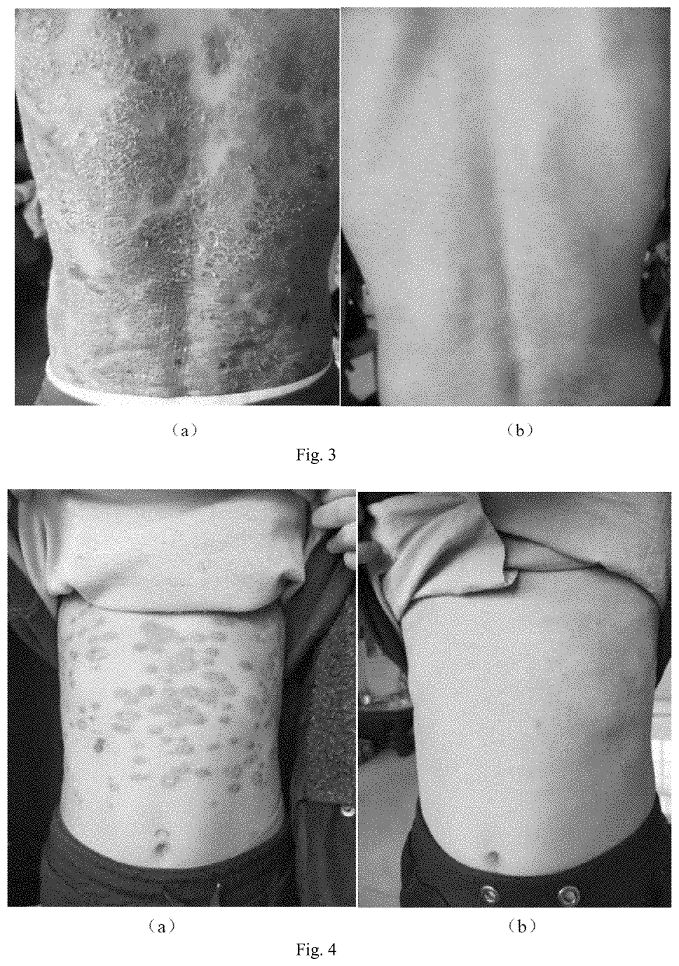

No. 3 patient, Zhen * shuai, (152 **** 5927, No. 7, Fangzhuang, Peixian Development Zone, Jiangsu Province), suffered from joint-type psoriasis for 2 and a half years, with purple tinea areas all over the body, especially, the joints of the extremities were prominent, and the conditions were serious. The skin turned back normal after taking the biological microbial drug for 3 months, and complete recovery was achieved after consolidation for a month. The skin conditions before and after treatment are shown in FIG. 3.

Clinical Case 4

No. 4 patient, Ding * liang, (158****6569, Dinglou Village, Zhaozhuang Town, Feng County, Jiangsu Province), suffered from psoriasis vulgaris on the head, chest, back, buttocks and thighs and legs for 3 years, and complete recovery was achieved after taking the microbial drug for 4 months, and consolidation therapy for another month. The skin conditions before and after treatment are shown in FIG. 4.

Clinical Case 5

No. 5 patient, daughter of Zhang * yan (134****2164, Zhangzhuang Village Xuzhai Town, Shan County, Shandong Province), suffered from pustular psoriasis for 15 years, with scald head up to 0.5 cm thick all around, without any healthy area all over the body. The psoriasis is thick and sticky, brown in color, with pus and bleeding. The patient was up to 39-41° C. in severe cases. Over the years, the patient have been seeking medical advices all around, such as Beijing 301 Hospital, Shanghai Changhai Dermatology Hospital as well as Wuhan, Shijiazhuang and Shandong Skin Hospitals, and Jining Medical College Affiliated Hospital, etc., but the patient lost confidence in recovery, even the most basic control cannot be achieved. Out of desperation, the patient found our biological microbial drug. After 4 months, the skin turned back shiny as it was, and complete recovery was achieved in 6 months.

From the first patient taking biological microbial drug in 2010 until Jul. 09, 2019, the total number of psoriasis patients taking biological microbial drug is about 400, and generally significant effective was achieved after about 15 days of taking the drug, and the effect was prominent in 30 days. The effective rate is 100%, and the recovered rate is over 90%.

Clearly, compared with other existing drugs, the drug for treating psoriasis provided by this invention has the advantages of quick and effective treatment of psoriasis, without toxic or side effects, less relapse, convenient medication, and low cost, etc.

What is claimed is:

1. A biological microbial drug for treating skin diseases, comprising: Pleurotus ostreatus cultivated in a Pleurotus ostreatus medium added with a traditional Chinese medicine (TCM) combination, wherein, the TCM combination comprises Sangusis draconis, Astragalus membranaceus, Schisandra chinensis, Herba epimedii, Eucommia ulmoides, Chinese wolfberry and Cortex dictamni.

2. The biological microbial drug for treating the skin diseases according to claim 1, wherein a weight ratio of the TCM combination to the Pleurotus ostreatus medium is (89.5 -110.5):(162-324), and the Pleurotus ostreatus medium added with the TCM combination has a water content of 55%-65% and a pH value of 6.5-8.

3. The bacteriological microbial drug for treating the skin diseases according to claim 1, wherein the TCM combination comprises, by weight, 3.5%-6.5% of the Sangusis draconis, 18.5%-21.5% of the Astragalus membranaceus, 13.5%-16.5% of the Schisandra chinensis, 8.5%-11.5% of the Herba epimedii, 3.5%-6.5% of the Eucommia ulmoides, 28.5%-31.5% of the Chinese wolfberry and 13.5%-16.5% of the Cortex dictamni.

4. The bacteriological microbial drug for treating the skin diseases according to claim 1, wherein the Pleurotus ostreatus medium comprises a cottonseed husk/broadleaf tree sawdust/corn core/bean straw powder/peanut shell/bamboo, a soybean cake powder, a maize meal, a bran, a lime powder, a gypsum powder and a brown sugar/white sugar, with a weight ratio of (100-200):(10-20):(20-40):(20-40):(6-12):(4-8):(2-4).

5. A method for preparing a biological microbial drug for treating the skin diseases, comprising the following steps:
1) preparing and sterilizing a Pleurotus ostreatus culture medium added with a TCM combination having a water content of 55%-65% and a pH value of 6.5-8, wherein the TCM combination comprises Sangusis draconis, Astragalus membranaceus, Schisandra chinensis, Herba epimedii, Eucommia ulmoides, Chinese wolfberry and Cortex dictamni, and the weight ratio of the TCM combination to the Pleurotus ostreatus medium is (89.5-110.5):(162-324);
2) under sterile conditions, inoculating a strain of Pleurotus ostreatus by a 4%-9% v/v inoculum size, with an incubation temperature below 30° C. and a humidity of 60%-70%;
3) keeping a humidity of 80%-95% when mushrooms appear; and
4) when growing to a harvesting standard, the Pleurotus ostreatus is harvested and dried in time to obtain the biological microbial drug.

6. The method for preparing the biological microbial drug for treating the skin diseases according to claim 5, wherein the Pleurotus ostreatus medium added with the TCM combination comprises 3.5-6.5 parts of the Sangusis draconis, 18.5-21.5 parts of the Astragalus membranaceus, 13.5-16.5 parts of the Schisandra chinensis, 8.5-11.5 parts of the Herba epimedii, 3.5-6.5 parts of the Eucommia ulmoides, 28.5-31.5 parts of the Chinese wolfberry, 13.5-16.5 parts of the Cortex dictamni, 100-200 parts of a cottonseed husk/broadleaf tree sawdust/corn core/bean straw powder/peanut shell/bamboo crumbs, 10-20 parts of a soybean cake powder, 20-40 parts of a maize meal, 20-40 parts of a bran, 6-12 parts of a lime powder, 4-8 parts of a gypsum powder and 2-4 parts of a brown sugar/white sugar.

7. A method for using the biological microbial drug for treating the skin diseases according to claim 1, comprising the following step: applying the biological microbial drug for treating the skin diseases.

8. The method according to claim 7, wherein the skin diseases comprise psoriasis, eczema, urticaria and neurodermatitis.

9. The method to claim 7, wherein the Pleurotus ostreatus is brewed or boiled with water and is taken once a day.

10. The method according to claim 7, wherein a weight ratio of the TCM combination to the Pleurotus ostreatus medium is (89.5-110.5):(162-324), and the Pleurotus ostreatus medium added with the TCM combination has a water content of 55%-65% and a pH value of 6.5-8.

11. The method according to claim 7, wherein the TCM combination comprises, by weight, 3.5%-6.5% of the Sangusis draconis, 18.5%-21.5% of the Astragalus membranaceus, 13.5%-16.5% of the Schisandra chinensis, 8.5%-11.5% of the Herba epimedii, 3.5%-6.5% of the Eucommia ulmoides, 28.5%-31.5% of the Chinese wolfberry and 13.5%-16.5% of the Cortex dictamni.

12. The method according to claim 7, wherein the Pleurotus ostreatus medium comprises a cottonseed husk/broadleaf tree sawdust/corn core/bean straw powder/peanut shell/bamboo, a soybean cake powder, a maize meal, a bran, a lime powder, a gypsum powder and a brown sugar/white sugar, with a weight ratio of (100-200):(10-20):(20-40):(20-40):(6-12):(2-4).
